(19) United States
(12) Patent Application Publication
(10) Pub. No.: US 2007/0037818 A1
(43) Pub. Date: Feb. 15, 2007

(54) Discovery of valuable properties for the primary energy source "PES 2000 plus Folic Acid"

(76) Inventor: William Hubert Livingston, Artesia, NM (US)

Correspondence Address: William H. Livingston, D.V.M.;Livingston Research Corporation, R223 North 13th Street, Artesia, NM, 88210, US

(21) Appl. No.: 11/203,465

(22) Filed: Aug. 15, 2005

(51) Int. Cl. A61K 31/525; A61K 31/19

(52) U.S. Cl. 514/251; 514/574

(57) ABSTRACT

A. An oral therapeutic dose of “PES 2000 plus Folic Acid”, which contains folic acid, succinic acid, glutamic acid, calcium succinate, B-12, di-calcium phosphate, magnesium stearate, and gelatin. PES 2000 reduces formation of triglycerides, cholesterol, and thru oxidative metabolism excessive build up of apolipoprotein E4 is prevented. This reduction aids in the prevention and control of Alzheimer's Disease and Dementia. B. An oral therapeutic dose “PES 2000 plus Folic Acid” has been shown to lower blood sugar, triglycerides, cholesterol, A1c, and apolipoprotein levels, in type one and two diabetes. C. An oral therapeutic dose “PES 2000 plus Folic Acid” has been shown to reduce insulin resistance in animals and humans.

### BACKGROUND OF THE INVENTION

Method of Discovery is as Follows:

“Primary Energy Source, “PES 200 Plus Folic Acid”, containing a therapeutic dose, when taken by individual humans or animals, orally, did the following:

A. Increased energy by stimulating the energy cycle, the “Citric Acid Cycle”, and slowed aging in humans and animals, by decreasing oxidative radical production. This has been demonstrated in thousands of humans, horses, cats and dogs. This was demonstrated thru the use of Livingston Animal Clinic's. SR-38, a precursor of “PES 2000” B. Research work shows “PES 2000 plus Folic Acid” will help prevent and control Alzheimer's disease and Dementia.

C. A therapeutic dose of “PES 2000 plus Folic Acid” has been shown to be effective in preventing damage caused by diabetes and insulin resistance thru research conducted by Livingston Research Corporation (LRC) and Livingston Animal Clinic.

D. Other Kreb cycle metabolites may be substitute for succinic acid, but succinic acid is better, because it is smaller and more readily absorbed especially orally.

1. A therapeutic dose of“PES 2000 PLUS FOLIC ACID” aids in the prevention of damage caused by diabetes in humans and animals by lowering cholesterol, triglycerides, sugar levels, A1c and increased oxidative metabolism helps prevent insulin resistance.

2. A therapeutic dose of “PES 2000 PLUS FOLIC ACID”, helps anti-aging, Alzheimer's disease, and Dementia in humans.
